(19) United States
(12) Patent Application Publication
(10) Pub. No.: US 2013/0267712 A1
(43) Pub. Date: Oct. 10, 2013

(54) AROMATIC KETONE SYNTHESIS WITH AMIDE REAGENTS AND RELATED REACTIONS

(71) Applicants: Douglas A. Klumpp, Sycamore, IL (US); Erum K. Raja, DeKalb, IL (US)

(72) Inventors: Douglas A. Klumpp, Sycamore, IL (US); Erum K. Raja, DeKalb, IL (US)

(73) Assignee: Board of Trustees of Northern Illinois University, DeKalb, IL (US)

(21) Appl. No.: 13/826,870

(22) Filed: Mar. 14, 2013

Related U.S. Application Data
(60) Provisional application No. 61/619,311, filed on Apr. 2, 2012.

(51) Int. Cl. C07C 45/46; C07D 209/46; C07D 295/192; C07C 231/10; C07D 217/24; C07D 401/12

(52) U.S. Cl. CPC C07C 45/46; C07D 217/24; C07D 401/12; C07D 295/192; C07C 231/10; C07D 209/46
USPC 546/141; 568/309; 546/226; 564/184; 568/322; 568/323; 548/472

(57) ABSTRACT

A method of preparing an aryl carbonyl or aryl thiocarbonyl compound, comprises reacting an N-(nitroaryl)-amide or N-(nitroaryl)-thioamide with an aromatic ring, with a superacid catalyst, to produce the aryl carbonyl or aryl thiocarbonyl compound. The superacid is present in an amount of at most 8 equivalents in proportion to the N-(nitroaryl)-amide or N-(nitroaryl)-thioamide. A method of preparing aryl amide or aryl thioamide, comprises reacting an N-(nitroaryl)-carbamide or N-(nitroaryl)-thiocarbamide with an aromatic ring, with a superacid catalyst, to produce the aryl amide or aryl thioamide.

### REFERENCE TO RELATED APPLICATIONS

This application claims the benefit of U.S. Provisional Application No. 61/619,311 entitled “AROMATIC KETONE SYNTHESIS WITH AMIDE REAGENTS AND RELATED REACTIONS” filed Apr. 2, 2012 which is incorporated by reference in its entirety.

### FEDERALLY SPONSORED RESEARCH OR DEVELOPMENT

This invention was made with government support under GM085736-012A1 awarded by the National Institutes of Health, National Institute of General Medical Sciences. The government has certain rights in the invention.

### BACKGROUND

In 1877, Friedel and Crafts reported the synthesis of an aryl ketone with the use of a carboxylic acid chloride, aluminum chloride, and benzene. The Friedel-Crafts acylation may now be accomplished with carboxylic acids, as well as the carboxylic acid derivatives, esters and anhydrides. A wide variety of Lewis and Brønsted acids are also known to promote these electrophilic aromatic substitutions. The Friedel-Crafts acylation is a vitally important conversion for industry, as it is used to prepare chemical feedstock, synthetic intermediates, and fine chemicals.

Despite the long history of Friedel-Crafts acylation, there has been almost nothing reported in which amides are used in these conversions. The amide functional group is known for its low reactivity in acyl-transfer reactions. The poor electrophilic reactivity of amides is thought to arise from electron donation from the amide nitrogen into the carbonyl anti-bonding orbital (n_{N}→π*_{CO}), often depicted as the zwitterionic amide resonance structure. Because of poor electrophilic reactivity, amides have only been known to react with moderately strong nucleophiles. Many Friedel-Crafts acylations are also thought to occur via reactive acyl cation intermediates. However, the strong carbon-nitrogen bond in amides inhibits cleavage to acyl cations under acidic conditions. This has effectively prevented amides from being used in Friedel-Crafts acylation.

Though amides are generally not considered viable substrates for the Friedel-Crafts synthesis of aromatic ketones, recent studies have shown that destabilized amides can give these products in good yields. For example, β-lactams were shown to give aryl ketones from Friedel-Crafts reactions, however, these reactions are clearly driven by the release of strain in the β-lactam ring system. Recently described were also several examples of Friedel-Crafts acylation using amides. The chemistry utilized a Brønsted superacid (CF₃SO₃H) and a mechanism was proposed with dicationic superelectrophiles in the transformations. Presumably, these conversions are driven by the repulsive interaction of cationic charge centers in the dicationic intermediates. A recent report has also shown dramatically increased amide hydrolysis rates (nucleophilic attack by water) induced by decoupled resonance of the amide through torsional strain.

These previous studies prompted the search for a general synthetic route using amides in Friedel-Crafts chemistry, especially one in which amide resonance were to be diminished or eliminated. This lead to a recent report of superacid-assisted acyl transfers with arenes and amides (D. J. De Schepper, Thesis, December 2008). Particularly interesting were the use of N-arylamides, specifically N-(4-nitro-phenyl)-amides, for example N-(4-nitro-phenyl)-acetamide, N-(4-nitro-phenyl)-benzamide and 4-nitrophenyl isocyanate, in reactions with benzene and related compounds, using triflic acid to catalyze the reaction. These reactions produced the desired Friedel-Crafts acylation product and 4-nitroaniline. The triflic acid may easily be recovered for reuse as a catalyst, and the 4-nitroaniline may also be easily recovered and converted back into an N-arylamide. However, even though the triflic acid acts as a catalyst, all the reaction were carried out using more than 10 equivalents of triflic acid (typically 14-230 equivalents). The sole exception was an intramolecular acylation starting with a N-(2,4-dinitro-phenyl)-amide using 8.7 equivalents of triflic acid.

### SUMMARY

In a first aspect, the present invention is a method of preparing an aryl carbonyl or aryl thiocarbonyl compound, comprising reacting an N-(nitroaryl)-amide or N-(nitroaryl)-thioamide with an aromatic ring, with a superacid catalyst, to produce the aryl carbonyl or aryl thiocarbonyl compound. The superacid is present in an amount of at most 8 equivalents in proportion to the N-(nitroaryl)-amide or N-(nitroaryl)-thioamide.

In a second aspect, the present invention is a method of preparing aryl amide or aryl thioamide, comprising reacting an N-(nitroaryl)-carbamide or N-(nitroaryl)-thiocarbamide with an aromatic ring, with a superacid catalyst, to produce the aryl amide or aryl thioamide.

### DEFINITIONS

Equivalents refers to the ratio of moles of a later mentioned chemical used in a chemical reaction with respects to the moles of the first chemical mentioned. For example, the phrase “the reaction of N-(4-nitrophenyl)-acetamide with benzene in 4 equivalents CF₃SO₃H” means that the reaction included 4 moles of CF₃SO₃H for each mole of N-(4-nitrophenyl)-acetamide.

Superacid as used herein refers to Brønsted superacids having acidities higher than 100% sulfuric acid, having an Hₒ≦−12 on the Hammett scale. See FIG. 1. Examples include triflouoromethanesulfonic acid (TfOH, CF₃SO₃H, or triflic acid; Hₒ=−14.1), fluorosulfuric acid (FSO₃H; Hₒ=−15.1), FSO₃H—SbF₅ (magic acid; Hₒ=−27), CF₃SO₃H—SbF₅, HF—BF₃, HF—SbF₅, and solid superacids including Nafion H and CF₃(CF₂)₉SO₃H.

An aromatic ring or aryl group refers to any monovalent aromatic carbocyclic or heteroaromatic group, preferably of 3 to 10 carbon atoms. The aromatic ring or aryl group can be monocyclic (for example, phenyl (or Ph)) or polycyclic (for example, naphthyl) and can be unsubstituted or substituted. Preferred aryl groups include phenyl, naphthyl, furyl, thienyl, pyridyl, indolyl, quinolinyl or isoquinolinyl.

An aryl carbonyl or aromatic carbonyl is a compound which contains a carbonyl group (C═O) directly attached to an aromatic ring. Such compounds include ketones, aldehydes and amides, such acetophenone, benzaldehyde and benzophenone. Similarly, an aryl thiocarbonyl or aromatic thiocarbonyl is a compound which contains a thiocarbonyl group (C═S) directly attached to an aromatic ring.

An N-(nitroaryl)-amine is a compound which contains a nitro (NO₂) group directly attached to an aromatic ring, and a nitrogen directly attached to the aromatic ring.

An N-(nitroaryl)-amide is a compound which contains a nitro (NO₂) group directly attached to an aromatic ring, and the nitrogen of an amide (N—C═O) moiety directly attached to the aromatic ring. N—C═O moiety includes isocyanate (N═C═O), and carbamides (N—(C═O)—N). Preferably, a nitro group is attached at the 2 position (ortho or o-), the 3 position (meta or m-) or the 4 position (para or p-) relative to the amide when the aromatic ring is a phenyl group. Two or more nitro groups may be attached to the aromatic ring. Similarly, an N-(nitroaryl)-thioamide is a compound which contains a nitro (NO₂) group directly attached to an aromatic ring, and the nitrogen of a thioamide (N—C═S) moiety directly attached to the aromatic ring; N—C═S moiety includes isothiocyanate (N═C═S), and thiocarbamides (N—(C═S)—N).

Alkyl (or alkyl- or alk-) refers to a substituted or unsubstituted, straight, branched or cyclic hydrocarbon chain, preferably containing from 1 to 20 carbon atoms. More preferred alkyl groups are alkyl groups containing from 7 to 16 carbon atoms. Preferred cycloalkyls have from 3 to 10, preferably 3 to 6, carbon atoms in their ring structure. Suitable examples of unsubstituted alkyl groups include methyl, ethyl, propyl, isopropyl, cyclopropyl, butyl, iso-butyl, tert-butyl, sec-butyl, cyclobutyl, pentyl, cyclopentyl, hexyl, and cyclohexyl. Alkylaryl and alkylheterocyclic groups are alkyl groups covalently bonded to an aryl or heterocyclic group, respectively.

Alkenyl refers to a substituted or unsubstituted, straight, branched or cyclic, unsaturated hydrocarbon that contains at least one double bond, and preferably 2 to 20, more preferably 7 to 16, carbon atoms. Exemplary unsubstituted alkenyl groups include ethenyl (or vinyl)(—CH CH═CH₂), 1-propenyl, 2-propenyl (or allyl)(—CH₂—CH═CH₂), 1,3-butadienyl (—CH═CHCH═CH₂), 1-butenyl (—CH═CHCH₂CH₃), hexenyl, pentenyl, 1,3,5-hexatrienyl, and the like. Preferred cycloalkenyl groups contain 5 to 8 carbon atoms and at least one double bond. Examples of cycloalkenyl groups include cyclohexadienyl, cyclohexenyl, cyclopentenyl, cycloheptenyl, cyclooctenyl, cyclohexadienyl, cyclopentadienyl, and cyclooctatrienyl.

Substituted means that the moiety contains at least one, preferably 1 to 3, substituent(s). Suitable substituents include hydroxyl (—OH), amino (—NH₂), oxy (—O—), carbonyl (—CO—), thiol, alkyl, alkenyl, alkynyl, alkoxy, halo, nitrile, nitro, aryl and heterocyclic groups. These substituents can optionally be further substituted with 1 to 3 substituents. Examples of substituted substituents include carboxamide, alkylmercapto, alkylsulphonyl, alkylamino, dialkylamino, carboxylate, alkoxycarbonyl, alkylaryl, aralkyl, and alkylheterocyclic. Also included as substituents are solid supports, surfaces and polymers, for example polystyrene beads and particles, and glass surfaces.

A pharmaceutical compound is an organic compound which has a biological effect and may be used to treat or prevent a disease or condition. Examples include monoamine transport inhibitors (for example indatraline and sertraline), analgesics (for example naproxen), antihypertensives, and antibiotics.

A monomer is a compound which may be polymerized, for example styrene. A polymer is the product produced by polymerizing one or more monomers. Examples include polycarbonates, polyesters, polyamides, polyketones, polystyrene and polyurethanes.

### BRIEF DESCRIPTION OF THE DRAWINGS

FIG. 1 illustrates the range of acidities on Hammett scale of various superacids. The solid bars indicate measurements using indicators, and the broken line indicates an estimated by kinetics measurements. The numbers in parentheses are an indication of the percent mole content of the Lewis acid involved.

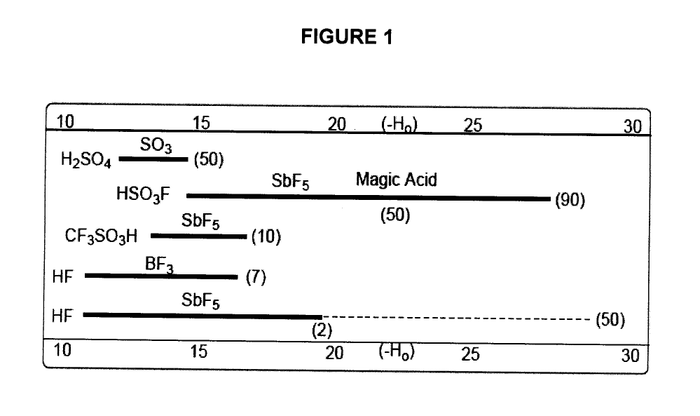

### DETAILED DESCRIPTION

The present invention makes use of the discovery of a general synthetic methodology using amides to prepare aryl carbonyls, such as aryl ketones. The chemistry is shown to be effective in intramolecular and intermolecular reactions. In some cases, the acylation is comparable—or better than—similar reactions with carboxylic acid chlorides. Moreover, the chemistry is done with a recyclable Brønsted acid and recoverable amine component. The methodology is also shown to be a useful route to aromatic amides from urea substrates—an unprecedented type of electrophilic aromatic substitution.

Amides and thiamides are designed with one or more electron-withdrawing nitro-substituents on the aryl-anilino group, to weaken the amide resonance and provide acyl transfer chemistry. Similarly, the formation of cyclic aromatic ketones is demonstrated. Furthermore, synthetic access to aromatic amides and thioamides is provided, including cyclic aromatic amides and thioamides, by the reactions of suitable aromatic ureas. The aromatic anilines may be recovered in high yields, providing the basis for a completely recyclable process, as the aromatic amide precursors may be prepared from thermal dehydration of the carboxylic acid derivative and the aromatic aniline. Moreover, the superacid catalyst may itself be quantitatively recycled by published procedures (B. L. Booth and T. A. El-Fekky, J. Chem. Soc. Perkin I 2441-2446 (1979)). Thus, an environmentally benign route to aromatic ketones and amides is provided.

In one aspect, the methodology uses an N-(nitroaryl)-amide or N-(nitroaryl)-thioamide to acylate an aromatic ring, to produce an aryl carbonyl or aryl thiocarbonyl product, respectively, and the N-(nitroaryl)-amine, using a superacid as a catalyst. Unlike prior reactions, the superacid is present in an amount of at most 8 equivalents.

In another aspect, the methodology uses N-(nitroaryl)-carbamide or N-(nitroaryl)-thiocarbamide to acylate an aromatic ring, to produce an aryl amide or aryl thioamide product, respectively, and the N-(nitroaryl)-amine, using a superacid as a catalyst. This unprecedented type of electrophilic aromatic substitution is preferably carried out with the superacid present in an amount of at most 16 equivalents, and in some cases at most 8 equivalents.

The N-(nitroaryl)-amide is preferably a N-(nitrophenyl)-amide, such as a N-(4-nitrophenyl)-amide, a N-(2-nitrophenyl)-amide, or a N-(2,4-dinitrophenyl)-amide, including compounds where the amide is isocyanate, and compounds of Formula (A):

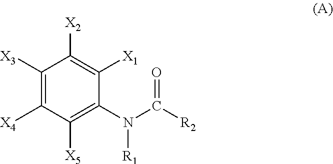

where R¹ is a substituent, preferably H or an alkyl containing 1-3 carbon atoms;
R² is a substituent, preferably substituted alkyl or aryl or NR³R⁴;
R³ and R⁴ are each independently a substituent, preferably substituted alkyl and/or aryl, optionally NR³R⁴ forming a ring; and
X¹, X², X³, X⁴, X⁵ are each independently H, or a substituent, where at least one of X¹, X², X³, X⁴, X⁵ is NO₂, more preferably, one or two of X¹, X², X³, X⁴, X⁵ is NO₂, and the remainder are H. Less preferably, R¹ and R² may be bonded together. In the case of an intramolecular acylation an aromatic ring will be part of R². In the case of intermolecular acylation, an aromatic ring is preferably not part of R². In the case of N-(nitroaryl)-carbamide, R² is NR³R⁴.

The N-(nitroaryl)-thioamide is preferably a N-(nitrophenyl)-thioamide, such as a N-(4-nitrophenyl)-thioamide, a N-(2-nitrophenyl)-thioamide, or a N-(2,4-dinitrophenyl)-thioamide, including compounds where the thioamide is isothiocyanate, and compounds of Formula (B):

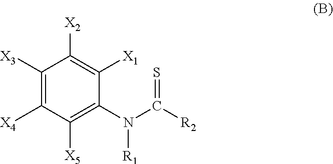

where R¹, R² and X¹, X², X³, X⁴, X⁵ each have the same meaning as in Formula (A). In the case of N-(nitroaryl)-thiocarbamide, R² is NR³R⁴.

The N-(nitroaryl)-amide and N-(nitroaryl)-thioamide may be prepared from the corresponding N-(nitroaryl)-amine by reaction of an appropriate carboxylic acid anhydride or acid chloride in the case of an amide, or an appropriate thiocarboxylic acid anhydride or acid chloride, or conversion from the appropriate amide using phosphorus pentasulfide or Lawesson's reagent, in the case of a thioamide. The N-(nitroaryl)-amide and N-(nitroaryl)-thioamide may also be prepared by dehydration of carboxylic acids or thiocarboxylic acides, as described by Hosseini-Sarvari, M. et al. (J. Org. Chem., 76, 2853 (2011)). The N-(nitroaryl)-amine, such as p-nitroaniline, may be recycled from a previous acylation reaction; such reuse of the N-(nitroaryl)-amine minimizes the waste products of the reaction.

In the case of an intermolecular acylation, the aromatic ring which is acylated may be substituted or unsubstituted, and in preferably a substituted phenyl ring, or benzene. In the case of an intramolecular acylation, the aromatic ring is a substituted aromatic ring, preferably substituted phenyl, which is part of R² of Formula (A) or (B).

The reaction may be carried out an appropriate temperature, for example 0 to 100° C. The reaction may be carried out without solvent (neat) or with a solvent, such as a chlorinated hydrocarbon, for example CH₂Cl₂.

In one aspect, unlike prior acylations using N-(nitroaryl)-amides and N-(nitroaryl)-thioamides, the superacid is present in an amount of at most 8 equivalents. Preferably, the reaction is intermolecular with the superacid present in an amount of at most 7, 6, 5 or 4 equivalents. Preferably, the reaction is intramolecular with the superacid present in an amount of at most 7, 6, 5 or 4 equivalents. The superacid may be reused from a previous acylation reaction. Preferably, the superacid is triflic acid, HF—BF₃, or a solid superacid.

In another aspect, acylations may be carried out using N-(nitroaryl)-thiocarbamide and N-(nitroaryl)-carbamide. The superacid is preferably present in an amount of at most 16 equivalents, including 15, 10, 9, 8, 7, 6, 5 or 4 equivalents. The reaction may be intermolecular or intramolecular. The superacid may be reused from a previous acylation reaction. Preferably, the superacid is triflic acid, HF—BF₃, or a solid superacid.

In another aspect, the acylations may be carried out using N-(nitroaryl)-amides and N-(nitroaryl)-thioamides attached to a solid support, such as a polystyrene bead or particle. This allows for ease of separation and recycling of the N-(nitroaryl)-amine product. Similarly, the acylations may be carried out using N-(nitroaryl)-thiocarbamide and N-(nitroaryl)-carbamide attached to a solid support for ease of separation and recycling of the N-(nitroaryl)-amine product.

The acylation reaction may be used to produce pharmaceutical compounds. For example, ketones 22 and 23 shown in Table 1, below, may be prepared as shown in the examples, and then using the reaction described in Walton et al. (J. Org. Chem. 76, 10011 (2011)) and Lee et al. (Chem. Med. Chem. 6, 321 (2011)) used in the preparation of the monoamine transport inhibitors, indatraline and sertraline. In another example, naproxen may be prepared by first preparing 1-(5-chloro-6-methoxy-2-naphthyl)-1-propanone, by acylation of 1-chloro-2-methoxy-naphthalene with N-(nitrophenyl) propanoyl amide, followed by further reaction as described in European Patent Application, Pub. No. 0 301 311 (1989). In yet another example, phenyl methyl thioether is acylated with N-(nitrophenyl) 3-methylbutanoyl amide, to form the 4-acetylated thioether; further reaction as described in U.S. Pat. No. 4,632,903 (1986) produces an antihypertensive drug. In yet another example, antibacterial phlorophenone derivatives may be prepared by acylation of a substituted phenyl compound using N-(nitrophenyl) propanoyl amide, similarly to the reaction described in European Patent Application, Pub. No. 0 069 536 (1983). Anti-inflammatory and analgesic 1-naphthylacetic acid derivatives may be prepared by acylation of methyl 1-naphthyl acetate with N-(nitrophenyl) propanoyl amide followed by reduction as described in U.S. Pat. No. 4,356,188 (1982).

The acylation reaction may be used to produce monomers, which then may optionally be used to prepare polymers. For example, acylation of styrene with N-(nitrophenyl) propanoyl amide, may be used to form a substituted styrene (a monomer) which may then in turn be polymerized into a polystyrene. Alternatively, styrene may be polymerized first, followed by acylation of the polystyrene. Trihaloacyl aromatics may be prepared by acylation of aromatics with N-(nitrophenyl) alkanoyl amide, where the alkyl is CX₃, (X═Cl or Br), which may be used as monomers in the preparation of polycarbonates, polyesters, polyamides, polyketones, and polyurathanes as described in European Patent Application, Pub. No. 0 189 266 (1986).

In summary, the present application presents a viable synthetic methodology for the use of amides in Friedel-Crafts acylation chemistry. The reactions have produced, for example aromatic ketones in high yields from intra- and intermolecular reactions. Conventional Friedel-Crafts reactions—with acid chlorides or anhydrides—often require excess quantities of Lewis acid (particularly AlCl₃) and they can produce significant amounts of corrosive vapor and aqueous aluminum waste. The present synthetic method is carried out with an acid that may be recycled quantitatively, while the amine component may also be recovered and recycled. Thus, this chemistry also minimizes the potential environmental impact of Friedel-Crafts acylations.

### EXAMPLES

The studies began with the reactions of N-(4-nitrophenyl)acetamide (1) and N-(4-nitrophenyl)benzamide (4) with benzene in CF₃SO₃H (triflic acid; eq 1, Scheme 1).

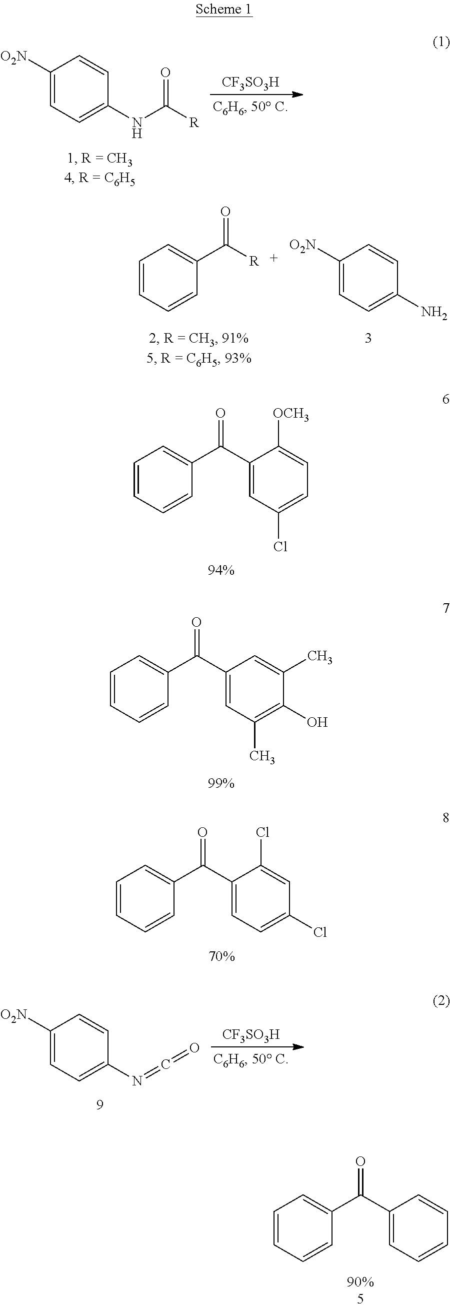

The reactions were conducted by adding 4.0 equivalents of CF₃SO₃H at 50° C. (3 hour reaction) to a 1:1 molar ratio of amide and arene dissolved in anhydrous CH₂Cl₂; after cooling to room temperature the resultant mixtures were poured over ice and water. Both amide substrates provided the desired aromatic ketones in excellent yields. For example, compound 1 gave acetophenone 2 in 91% yield. The byproduct, p-nitroaniline (3), may be recovered in greater than 80% yield and triflic acid may itself be quantitatively recycled. Since both components can be re-used, this Friedel-Crafts acylation represents a conversion producing little or no chemical waste. The benzamide derivative (4) likewise gives a good yield of the aromatic ketones (5-8) from the respective arenes (in the case of 8, 1,3-dichlorobenze was used at the solvent instead of CH₂Cl₂). The reaction of N-(2,4-dinitrophenyl) benzamide with benzene, also gave a 93% yield of 5. Furthermore, the reaction of 4 with cumene gave a 99% yield of the two isomers, which were not separated. These yields are comparable to synthetic reactions that use carboxylic acid chlorides or anhydrides leading to benzophenone (5) and related ketones. In this study, benzophenone (5) was prepared in good yield from direct reaction of the p-nitrophenylisocyanate (9) in CF₃SO₃H and benzene. Thus, the isocyanate 9 leads to formation of amide 4 and this reacts further to give benzophenone (5). In this conversion, compound 9 functions as a novel phosgene equivalent, as it provides only the carbonyl group in the final product 5.

The 4-nitrophenylamides (10-16) also underwent very efficient intramolecular reactions (Table 1).

TABLE 1
Reactant amides and product ketones from the reaction with CF₃SO₃H.

| Starting Material | | Product | | Yield^{a} |
|---|---|---|---|---|
| 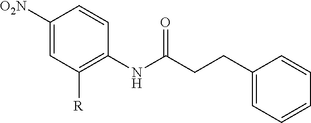 | 10 | 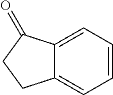 | 17 | 90%^{b} |
| 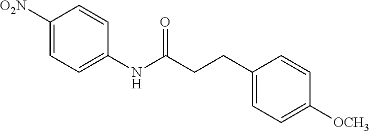 [SMILES: COc1ccc(CCC(=O)Nc2ccc([N+](=O)[O-])cc2)cc1] | 11 | 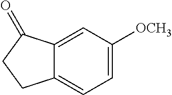 [SMILES: COc1ccc2c(c1)C(=O)CC2] | 18 | 75%^{b} |
| 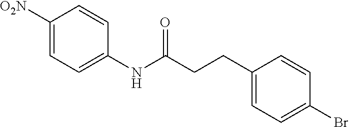 [SMILES: O=C(CCc1ccc(Br)cc1)Nc1ccc([N+](=O)[O-])cc1] | 12 | 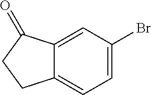 [SMILES: O=C1CCc2ccc(Br)cc21] | 19 | 88%^{b} |
| 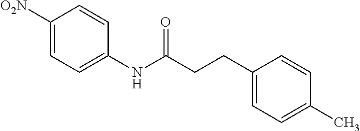 [SMILES: Cc1ccc(CCC(=O)Nc2ccc([N+](=O)[O-])cc2)cc1] | 13 | 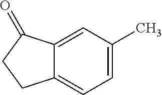 | 20 | 96%^{b} |
| 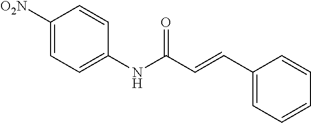 [SMILES: O=C(/C=C/c1ccccc1)Nc1ccc([N+](=O)[O-])cc1] | 14 | 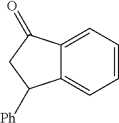 [SMILES: O=C1CC(c2ccccc2)c2ccccc21] | 21 | 94%^{c} |
| 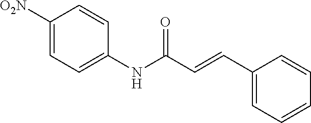 [SMILES: O=C(/C=C/c1ccccc1)Nc1ccc([N+](=O)[O-])cc1] | 15 | 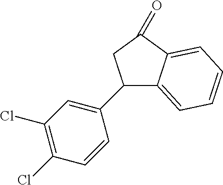 [SMILES: O=C1CC(c2ccc(Cl)c(Cl)c2)c2ccccc21] | 22 | 95%^{d} |
| 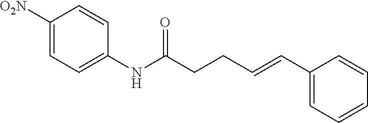 [SMILES: O=C(CC/C=C/c1ccccc1)Nc1ccc([N+](=O)[O-])cc1] | 16 | 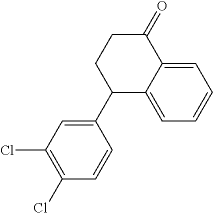 [SMILES: O=C1CCC(c2ccc(Cl)c(Cl)c2)c2ccccc21] | 23 | 76%^{d} |

^{a}Isolated yields. ^{b}Reaction with 4 eq CF₃SO₃H, CHCl₃, 50° C. ^{c}Reaction with 4 eq CF₃SO₃H, C₆H₆, 50° C. ^{d}Reaction with 4 eq CF₃SO₃H, o-C₆H₄Cl₂, 50° C.

For example, amide 10 (R═H) provided 1-indanone (17) in 90% yield upon reaction with CF₃SO₃H in CHCl₃. The 4-nitroaniline by-product may be isolated with up to 90% recovery. Other acids were studied in this conversion (H₂SO₄, CF₃CO₂H, AlCl₃, HY-zeolite, Sc(OTf)₃), but none successfully converted 10 to the indanone 17. Amides 11-13 also gave the corresponding substituted 1-indanones (18-20) in good yields. The amide 10′ (below), also gave the corresponding substituted 1-indanone 17′ (below), in 68% yield (other isomers were also obtained).

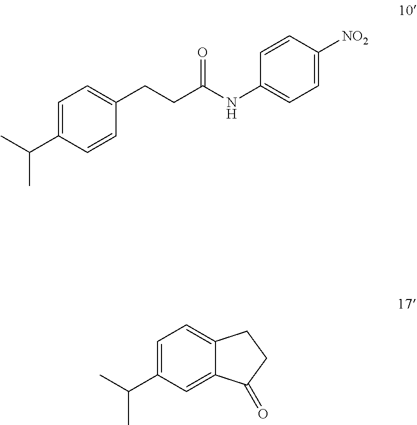 [SMILES: CC(C)c1ccc(CCC(=O)Nc2ccc([N+](=O)[O-])cc2)cc1 ; CC(C)c1ccc2c(c1)C(=O)CC2]

Unsaturated amides are known to undergo addition reactions with arenes via superelectrophilic intermediates. As such, arylation may be coupled with cyclization to give the aryl-substituted 1-indanones 21-22 (Table 1). A similar reaction may be accomplished with amide 16, providing access to the tetralone 23. Ketones 22 and 23 are synthetic intermediates used in the preparation of monoamine transport inhibitors, indatraline and sertraline. The reaction of 14 was carried out using benzene as the solvent.

Given the success of the Friedel-Crafts acylations, it was sought to determine if similar reactions could be accomplished with urea or carbamide substrates. The carbamides are even less reactive electrophiles (compared to the amide group) because the urea carbonyl group interacts with two adjacent nitrogen centers. However, it has now been found that nitro-substituted aryl groups do indeed activate carbamides for electrophilic aromatic substitution. Intermolecular reactions of carbamides 24 and 26 provided 3,4-dihydro-1(2H)-isoquinolinones (29-30) in good yields (Table 2).

TABLE 2
Products and yields from reactions of carbamides in CF₃SO₃H.

| Starting Material | | Product | | Yield^{a} |
|---|---|---|---|---|
| 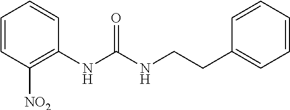 [SMILES: O=C(NCCc1ccccc1)Nc1ccccc1[N+](=O)[O-]] | 24 | 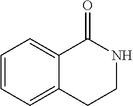 [SMILES: O=C1NCCc2ccccc21] | 29 | 98%^{b} |
| 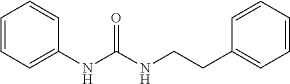 [SMILES: O=C(NCCc1ccccc1)Nc1ccccc1] | 25 | Quantitative recovery of 25 | | —^{b} |
| 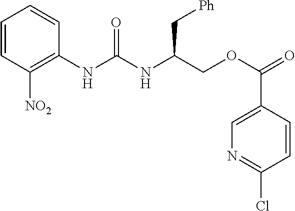 [SMILES: O=C(Nc1ccccc1[N+](=O)[O-])N[C@H](COC(=O)c1ccc(Cl)nc1)Cc1ccccc1] | 26 | 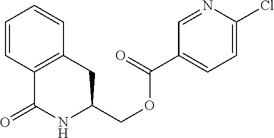 [SMILES: O=C(OC[C@@H]1Cc2ccccc2C(=O)N1)c1ccc(Cl)nc1] | 30 | 80%^{b} |
| 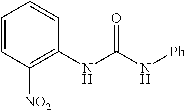 [SMILES: O=C(Nc1ccccc1)Nc1ccccc1[N+](=O)[O-]] | 27 | 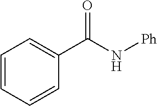 [SMILES: O=C(Nc1ccccc1)c1ccccc1] | 31 | 68%^{c} |
| 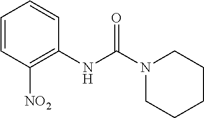 [SMILES: O=C(Nc1ccccc1[N+](=O)[O-])N1CCCCC1] | 28 | 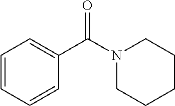 [SMILES: O=C(c1ccccc1)N1CCCCC1] | 32 | 52%^{c} |

^{a}Isolated yields. ^{b}Reaction with 8 eq CF₃SO₃H, CHCl₃, 50° C. ^{c}Reaction with 8 eq CF₃SO₃H, C₆H₆, 50° C.

In the case of carbamide 24, the product 3,4-dihydro-1(2H)-isoquinolinone (29) was formed in 98%, while the L-phenylalanol-derived carbamide (26) gave product (30) in 80% yield. Interestingly, reaction of urea 25 (under the same reactions conditions) does not give cyclized product 29, but the starting material was recovered. This demonstrates the importance of the nitro-substituent in the activation of the carbamide.

Carbamides 27 and 28 gave the aromatic amide products (31-32) by intermolecular reactions with excess benzene and CF₃SO₃H. In the case of 27, amide 31 was not formed in reactions with H₂SO₄ or CF₃CO₂H. Successful conversion of 27 to 31 only occurred with at least 8 equivalents of CF₃SO₃H. Up to 95% of the 2-nitroaniline can be recovered after the reaction.

Carbamide 28 (N-(2-nitrophenyl)piperidine-1-carboxamide) was also reacted toluene, p-xylene, and naphthalene as shown in Table 3, in CHCl₃ with 16 equivalents CF₃SO₃H at 50° C. for 12 hours; however the product of the reaction with naphthalene was lost up purification.

TABLE 3
Reaction of N-(2-nitrophenyl) piperidine-1-carboxamide with various
arenes.

| Arene | Product | Yield |
|---|---|---|
| 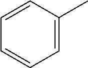 | 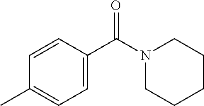 | 35% |
| 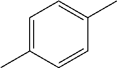 | 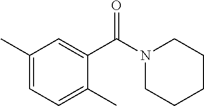 [SMILES: Cc1ccc(C)c(C(=O)N2CCCCC2)c1] | 20% |
| 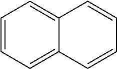 | 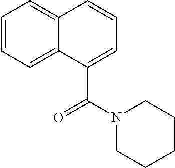 [SMILES: O=C(c1cccc2ccccc12)N1CCCCC1] | lost upon purification |

As shown in Scheme 2, reaction of carbamide 33 with CF₃SO₃H did not lead to the expect product 34, but rather to the observed product 35. To obtain the expected product, the alcohol was protected to form carbamide 26 before reaction, and then reaction product 30 was deprotected using sodium hydroxide in water, to form the desired product 34. Carbamide 33 was formed from a 1:1 mixture of 2-nitrophenyl isocyanate and L-phenylalaminol in anhydrous THF (prepared at 0° C.) followed by reaction for 12 hours at room temperature under argon. After purification, carbamide 33 was dissolved in anhydrous dichloromethane under argon inlet, triethylamine and 6-chloronicotinoyl chloride were added and the reaction left to stir for 12 hours at room temperature to form 26. The resulting mixture was then poured over ice/water and extracted using CHCl₃.

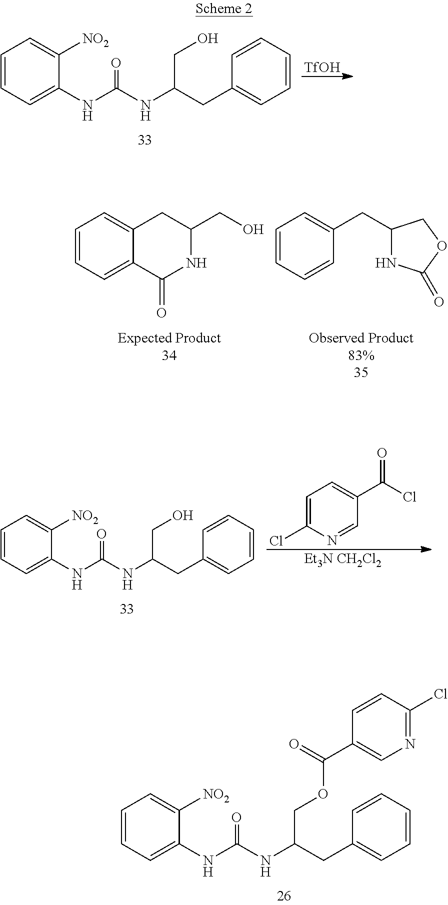

Additional intramolecular reactions of carbamides are shown in Table 4. Reactions were carried out in dichloromethane for 12 hours at 50° C. using 16 equivalent of CF₃SO₃H.

TABLE 4
Starting materials, products and yields from reactions of carbamides in
CF₃SO₃H.

| | | |
|---|---|---|
| 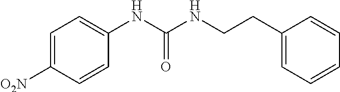 [SMILES: O=C(NCCc1ccccc1)Nc1ccc([N+](=O)[O-])cc1] | 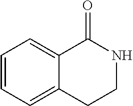 [SMILES: O=C1NCCc2ccccc21] | 85% |
| 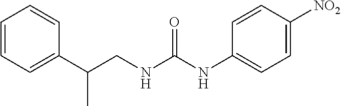 [SMILES: CC(CNC(=O)Nc1ccc([N+](=O)[O-])cc1)c1ccccc1] | 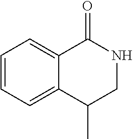 | 84% |
| 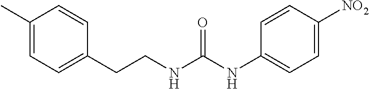 [SMILES: Cc1ccc(CCNC(=O)Nc2ccc([N+](=O)[O-])cc2)cc1] | 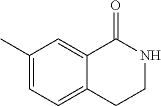 | 75% |
| 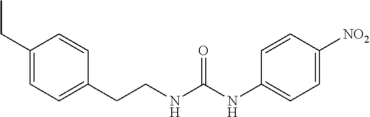 [SMILES: CCc1ccc(CCNC(=O)Nc2ccc([N+](=O)[O-])cc2)cc1] | 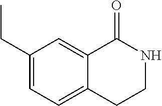 | 55% |
| 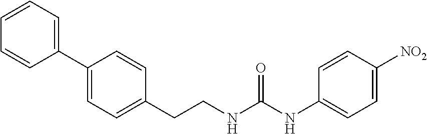 [SMILES: O=C(NCCc1ccc(-c2ccccc2)cc1)Nc1ccc([N+](=O)[O-])cc1] | 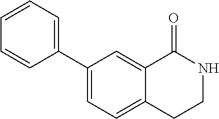 [SMILES: O=C1NCCc2ccc(-c3ccccc3)cc21] | 56% |
| 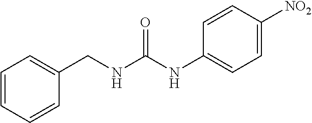 [SMILES: O=C(NCc1ccccc1)Nc1ccc([N+](=O)[O-])cc1] | 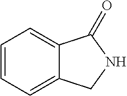 | 48% |

A number of carbamides (Table 5) were reacted with CF₃SO₃H, but underwent polymerization and/or decomposition, and no products were isolated.

TABLE 5
Carbamides that underwent polymerization and/or decomposition.

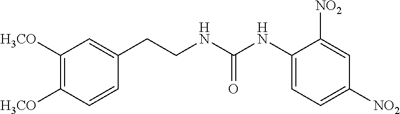 [SMILES: COc1ccc(CCNC(=O)Nc2ccc([N+](=O)[O-])cc2[N+](=O)[O-])cc1OC]

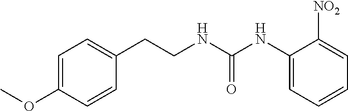 [SMILES: COc1ccc(CCNC(=O)Nc2ccccc2[N+](=O)[O-])cc1]

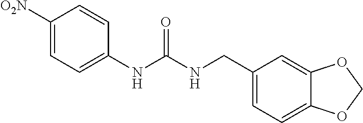 [SMILES: O=C(NCc1ccc2c(c1)OCO2)Nc1ccc([N+](=O)[O-])cc1]

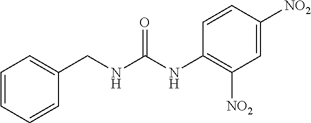 [SMILES: O=C(NCc1ccccc1)Nc1ccc([N+](=O)[O-])cc1[N+](=O)[O-]]

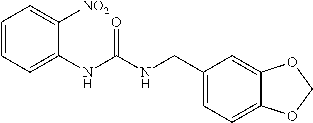 [SMILES: O=C(NCc1ccc2c(c1)OCO2)Nc1ccccc1[N+](=O)[O-]]

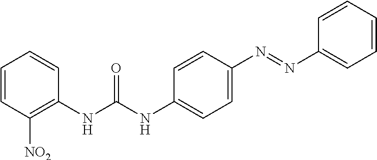 [SMILES: O=C(Nc1ccc(/N=N/c2ccccc2)cc1)Nc1ccccc1[N+](=O)[O-]]

With respects to a possible reaction mechanism, Friedel-Crafts acylation with carboxylic acid chlorides or anhydrides often occurs through acyl cation intermediates, however amides generally do not produce acyl cations in acidic media. Nevertheless, amide 4 can be reacted with CF₃SO₃H (4 equivalents) in CH₂Cl₂ without an arene nucleophile, and when the mixture is poured over ice, benzoic acid is isolated in 90% yield and no starting amide is found. This observation is consistent with the formation of either the benzoyl cation or the mixed anhydride with the triflate anion.

### REFERENCES

(1) C. Friedel, J. M. Crafts Compt. Rend. 84, 1450 (1877).
(2) P. H. Gore in Friedel-Crafts and Related Reactions; G. A. Olah, Ed.; John Wiley & Sons Inc.: London, 1964, Vol. III, Part I, p. 1.
(3) G. Sartori, R. Maggi Chem. Rev. 111, PR181—PR214, (2011).
(4) H. G. Frank Industrial Aromatic Chemistry; Springer: Berlin, 1988.
(5) The Amide Linkage, A. Greenberg, C. M. Breneman, J. F. Liebman, Eds.; John Wiley & Sons Inc.: New York, 2000.
(6) S. A. Glover, J. M. White, A. A. Rosser, K. M. Digianantonio J. Org. Chem. 76, 9757 (2011).
(7) G. A. Olah, A. M. White J. Am. Chem. Soc. 89, 7072 (1967).
(8) K. W. Anderson, J. J. Tepe Org. Lett. 3, 459 (2002).
(9) D. A. Klumpp, R. Rendy, Y. Zhang, A. Gomez, A. McElrea, Org. Lett. 6, 1789 (2004).
(10) M. Hutchby, C. E. Houlden, M. F. Haddow, S, N. G. Tyler, G. C. Lloyd-Jones, K. I. Booker-Milburn Angew. Chem. Int. Ed. 51, 548-551 (2012).
(11) B. L. Booth, T. A. El-Fekky J. Chem. Soc. Perkin I 2441-2446 (1979).
(12) Olah, G. A., Reddy, V. P. and Prakash, G. K. S. 2000. Friedel-Crafts Reactions. Kirk-Othmer Encyclopedia of Chemical Technology. Vol. 12, 159-199.
(13) D. Gauvreau, S. J. Dolman, G. Hughes, P. D. O'Shea, I. W. Davies J. Org. Chem. 75, 4078 (2010).
(14) F. Effenberger, R. Gleiter Chem. Ber. 97, 472 (1964).
(15) K. Yu. Koltunov, S. Walspurger, J. Sommer Eur. J. Org. Chem. 2004, 4039 (2004).
(16) J. G. A. Walton, D. C. Jones, P. Kiuru, A. J. Durie, N. J. Westwood, A. H. Fairlamb Chem. Med. Chem. 6, 321-328 (2011).
(17) S. H. Lee, I. S. Kim, Q. R. Li, G. R. Dong, L. S. Jeong, Y. H. Jung J. Org. Chem. 76, 10011-10019 (2011).
(18) G. A. Olah, D. A. Klumpp, Superelectrophiles and Their Chemistry (Wiley, New York, 2008).
(19) T. Ohta, K. Shudo, T. Okamoto Tetrahedron Lett. 25, 325-329 (1984).
(20) C. Cox, T. Lectka Acc. Chem. Rev. 33, 849-858 (2000).
(21) D. J. De Schepper, CHEMISTRY INVOLVING SUPERELECTROPHILES: SUPERACID ASSISTED CYCLIZATIONS AND BENZENE ELIMINATION OF NITRO-POLYAROMATIC HYDROCARBONS AND SUPERACID ASSISTED ACYL TRANSFERS WITH ARENES AND AMIDES (Thesis, Department of Chemistry and Biochemistry, Northern Illinois University, De Kalb, Ill., December 2008) UMI Number 1460942.
(22) A. M. Barrios, S. M. Lippard J. Am. Chem. Soc. 121, 11751 (1999).
(23) European Patent Application, Pub. No. 0 301 311 (1989).
(24) U.S. Pat. No. 4,632,903 (1986).
(25) European Patent Application, Pub. No. 0 069 536 (1983).
(26) U.S. Pat. No. 4,356,188 (1982).
(27) European Patent Application, Pub. No. 0 189 266 (1986).
(28) Hosseini-Sarvari, M., Sodagar, E. and Doroodmand, M. M., Journal of Organic Chemistry, 76, 2853 (2011).

1. A method of preparing an aryl carbonyl or aryl thiocarbonyl compound, comprising: reacting an N-(nitroaryl)-amide or N-(nitroaryl)-thioamide with an aromatic ring, with a superacid catalyst, to produce the aryl carbonyl or aryl thiocarbonyl compound,
wherein the superacid is present in an amount of at most 8 equivalents in proportion to the N-(nitroaryl)-amide or N-(nitroaryl)-thioamide.

2. The method of claim 1, wherein the aromatic ring is not attached to the N-(nitroaryl)-amide or N-(nitroaryl)-thioamide.

3. The method of claim 1, wherein the aromatic ring is attached to the N-(nitroaryl)-amide or N-(nitroaryl)-thiamide, and the superacid is present in an amount of at most 7 equivalents in proportion to the N-(nitroaryl)-amide or N-(nitroaryl)-thioamide.

4. The method of claim 1, wherein the aryl carbonyl compound is produced.

5. The method of claim 1, wherein the aryl thiocarbonyl compound is produced.

6. The method of claim 1, wherein the superacid is present in an amount of at most 7 equivalents in proportion to the N-(nitroaryl)-amide or N-(nitroaryl)-thioamide.

7. The method of claim 1, wherein the superacid is present in an amount of at most 4 equivalents in proportion to the N-(nitroaryl)-amide or N-(nitroaryl)-thioamide.

8. The method of claim 4, wherein the N-(nitroaryl)-amide is an N-(nitrophenyl)-amide.

9. The method of claim 1, further comprising recovering p-nitroaniline produced during the reaction.

10. The method of claim 1, wherein the aromatic ring is a substituted phenyl group or benzene.

11. The method of claim 1, wherein the reaction produces an aryl carbonyl compound, and the aryl carbonyl compound is a ketone.

12. The method of claim 1, wherein the reaction produces an aryl carbonyl compound, and the aryl carbonyl compound is an amide.

13. The method of claim 1, wherein the reacting is reacting the N-(nitroaryl)-amide with an aromatic ring, and the N-(nitroaryl)-amide is selected from the group consisting of 4-nitrophenyl isocyanate, N-(4-nitrophenyl)-acetamide and N-(4-nitrophenyl)-benzamide.

14. The method of claim 1, wherein the superacid is triflic acid or HF—BF₃.

15. The method of claim 1, further comprising isolating the superacid after the reacting.

16. The method of claim 15, further comprising carrying out another reacting with the superacid as a catalyst.

17. The method of claim 15, further comprising recovering p-nitroaniline produced during the reaction.

18. The method of claim 17, further comprising forming a N-(nitroaryl)-amide from the p-nitroaniline.

19. The method of claim 2, further comprising recovering p-nitroaniline produced during the reaction.

20-22. (canceled)

23. A method of preparing aryl amide or aryl thioamide, comprising: reacting an N-(nitroaryl)-carbamide or N-(nitroaryl)-thiocarbamide with an aromatic ring, with a superacid catalyst, to produce the aryl amide or aryl thioamide.

24-40. (canceled)
